(19) United States
(12) Patent Application Publication
(10) Pub. No.: US 2023/0105167 A1
(43) Pub. Date: Apr. 6, 2023

(54) METHODS FOR GENETIC IDENTIFICATION AND RELATEDNESS DETECTION

(71) Applicant: The Regents of the University of California, Oakland, CA (US)

(72) Inventors: Richard E. Green, Santa Cruz, CA (US); Remy Nguyen, Santa Cruz, CA (US)

(21) Appl. No.: 17/944,855

(22) Filed: Sep. 14, 2022

Related U.S. Application Data
(60) Provisional application No. 63/244,118, filed on Sep. 14, 2021.

(51) Int. Cl. G16B 20/20; G16B 20/40; G16B 30/00

(52) U.S. Cl. CPC G16B 20/20; G16B 20/40; G16B 30/00

(57) ABSTRACT

Provided are computer-implemented methods for comparing genotype data from a first sample to a limited amount of DNA sequence data from a second sample. In certain embodiments, the first sample is from a known individual and the second sample is an unknown sample. The methods find use in a variety of contexts, including for genetic identity detection, e.g., for forensic and other applications. Also provided are computer-implemented methods for assessing the degree of relatedness between genotype data from a first sample and a limited amount of DNA sequence data from a second sample. Computer-readable media and systems that find use in practicing the methods of the present disclosure are also provided.

### CROSS-REFERENCE TO RELATED APPLICATIONS

This application claims the benefit of U.S. Provisional Patent Application No. 63/244,118, filed Sep. 14, 2021, which application is incorporated herein by reference in its entirety.

### STATEMENT OF GOVERNMENT SUPPORT

This invention was made with Government support under contract A21-0538-001 awarded by National Institute of Justice. The Government has certain rights in the invention.

### INTRODUCTION

DNA-based identification in forensics is typically accomplished via genotyping allele length at a defined set of short tandem repeat (STR) loci via PCR [1]. These PCR assays are robust, reliable, and inexpensive [2]. Given the multiallelic nature of each of these loci, a small panel of STR markers can provide suitable discriminatory power for personal identification [3, 4].

Massively parallel sequencing (MPS) technologies and genotype array technologies invite new approaches for DNA-based identification. Application of these technologies has provided catalogs of global human genetic variation at single-nucleotide polymorphic (SNP) sites and short insertion-deletion (INDEL) sites. For example, from the 1000 Genomes Project [5], there is now a catalog of nearly all human SNP and INDEL variation down to 1% worldwide frequency.

Genotype files, generated via MPS or genotype array, can be compared between individuals to find regions that are co-inherited or identical-by-descent (IBD) [6-8]. These comparisons are the basis of the relative finder functions in many direct-to-consumer genetic testing products [9, 10]. A special case of relative-finding is self-identification. This is a trivial comparison of genotype files as self comparisons will be identical across all sites, minus the error rate of the assay.

For many forensic samples, however, the available DNA may not be suitable for PCR-based STR amplification [11], genotype array analysis [12], or MPS to the depth required for comprehensive, accurate genotype calling [13]. In the case of PCR, one of the most common failure modes occurs when DNA is too fragmented for amplification. For these samples, it may be possible to directly observe the degree of DNA fragmentation from the decreased amplification efficiency of larger STR amplicons from a multiplex STR amplification [14]. In the case of severely fragmented samples, where all DNA fragments are shorter than the shortest STR amplicon length, PCR simply fails with no product.

### SUMMARY

Provided are computer-implemented methods for comparing genotype data from a first sample to a limited amount of DNA sequence data from a second sample. In certain embodiments, the first sample is from a known individual and the second sample is an unknown sample. The methods find use in a variety of contexts, including for genetic identity detection, e.g., for forensic and other applications. Also provided are computer-implemented methods for assessing the degree of relatedness between genotype data from a first sample and a limited amount of DNA sequence data from a second sample. Computer-readable media and systems that find use in practicing the methods of the present disclosure are also provided

### BRIEF DESCRIPTION OF THE FIGURES

FIG. 1: IBDGem schematic. Comparisons are made between the known genotype of a subject person (Person of Interest; blue) and low-coverage sequence data from a DNA sample. The probability of the observed data can be calculated under the model that the subject person carries the same two chromosomes as the person from whom the DNA sample is collected, i.e., is IBD2 (top). Similarly, the probability of the observed data can be calculated under the model that the subject person is genetically unrelated at this genome region, i.e., IBD0 (bottom). In this way, a log-likelihood ratio of these two models is generated. Not shown is the IBD1 model wherein the subject genotype and questioned data share one chromosome.

FIG. 2: IBDGem performance at various levels of genome sequence coverage. LLRs are aggregated across 200 SNPs. Top panel: each individual from the GBR panel was compared against itself (same individual comparisons) or a random non-self GBR individual (different individual comparisons) following down-sampling of sequence data to 2×, 1×, 0.5×, 0.1×, and 0.01×genome coverages. Bottom panel: analogous comparisons amongst individuals in the LWK panel.

FIG. 3: IBDGem performance using various population background allele frequency models. LLRs are aggregated across 200 SNPs. Top panel: each individual from the GBR panel was compared against itself (same individual comparisons) or a random non-self GBR individual (different individual comparisons) using allele frequencies from the indicated superpopulation as the background panel. EUR=European, AMR=American, SAS=South Asian, EAS=East Asian, AFR=African, LWK=Luhya, GBR=British. Bottom panel: analogous comparisons amongst individuals in the LWK panel.

FIG. 4: IBDGem self and non-self comparisons of GBR and LWK individuals at GSA genotype array sites, down-sampled to 1-fold coverage.

FIG. 5: IBDGem comparisons using DNA from hair. Top: Fold genome coverage distribution at known variable sites on chromosome 1 from hair panel. Illumina libraries were sequenced to similar depths. Variation in fold coverage represents the variability of DNA presence and recovery in human hairs. Bottom: IBDGem self (top in each panel) and non-self (bottom in each panel) comparisons using DNA data from hair and genotype arrays. Left panel is lower-coverage samples (<1×). Right panel is higher-coverage samples (>1×).

FIG. 6: IBDGem comparisons between related individuals in the MXL panel. Results of IBDGem at 1-fold down-sampled coverage followed by HiddenGem to apportion each genomic segment into IBD0, IBD1, or IBD2 states amongst annotated pedigrees.

FIG. 7A: Distribution of aggregated LLRs between IBD2 and IBD0 for self (orange) and non-self (blue) comparisons of GBR individuals with the input sequence data down-sampled to 2×, 1×, 0.5×, 0.1×, and 0.01× coverages. Lower and upper whiskers are set at 10th and 90th percentiles, respectively. Any underflow in the data is addressed by setting the affected LLR to the next representable value after 0.

FIG. 7B: Distribution of aggregated LLRs between IBD2 and IBD0 for self (orange) and non-self (blue) comparisons of LWK individuals with the input sequence data down-sampled to 2×, 1×, 0.5×, 0.1×, and 0.01× coverages. Lower and upper whiskers are set at 10th and 90th percentiles, respectively. Any underflow in the data is addressed by setting the affected LLR to the next representable value after 0. Note that the difference in LLRs between self and non-self comparisons for individual NA19374 is larger than for the rest of the panel, especially at 2×, 1×, and 0.5×. This is likely because the original sequence depth of NA19374 is twice as high as that of other samples (˜60× versus ˜30×), resulting in an actual coverage that is two times higher than the target after IBDGem's automated down-sampling process. With more sequence data, the program can more easily distinguish between IBD0 and IBD2 states, leading to a larger difference in LLRs.

FIG. 7C: Distribution of aggregated LLRs between IBD2 and IBD0 for self (orange) and non-self (blue) comparisons of GBR individuals using background allele frequencies from the AFR, AMR, EAS, EUR, SAS superpopulations, as well as from a specific ‘wrong’ population (i.e. LWK). Compared sequence data are down-sampled to 1× coverage. Lower and upper whiskers are set at 10th and 90th percentiles, respectively. Any underflow in the data is addressed by setting the affected LLR to the next representable value after 0.

FIG. 7D: Distribution of aggregated LLRs between IBD2 and IBD0 for self (orange) and non-self (blue) comparisons of LWK individuals using background allele frequencies from the AFR, AMR, EAS, EUR, SAS superpopulations, as well as from a specific ‘wrong’ population (i.e., GBR). Compared sequence data are down-sampled to 1× coverage. Lower and upper whiskers are set at 10th and 90th percentiles, respectively. Any underflow in the data is addressed by setting the affected LLR to the next representable value after 0. Note that the difference in LLRs between self and non-self comparisons for individual NA19374 is larger than for the rest of the panel. This is likely because the original sequence depth of NA19374 is twice as high as that of other samples (˜60× versus ˜30×), resulting in an actual coverage that is two times higher than the target after IBDGem's automated down-sampling process. With more sequence data, the program can more easily distinguish between IBD0 and IBD2 states, leading to a larger difference in LLRs.

FIG. 8: IBDGem comparisons between related individuals in the YRI (Yoruba) panel, using only GSA sites. Compared sequence data are down-sampled to 1-fold coverage.

FIG. 9: Scatter plot of aggregated LLRs between IBD2 and IBD0 for self (orange) and non-self (blue) comparisons of GBR individuals. LLRs are aggregated over 500 SNPs across chromosome 1. Compared sequence data are down-sampled to 1× coverage. Any underflow in the data is addressed by setting the affected LLR to the next representable value after 0.

FIG. 10: Scatter plot of aggregated LLRs between IBD2 and IBD0 for self (orange) and non-self (blue) comparisons of LWK individuals. LLRs are aggregated over 500 SNPs across chromosome 1. Compared sequence data are down-sampled to 1× coverage. Any underflow in the data is addressed by setting the affected LLR to the next representable value after 0.

FIG. 11A-11C: Distribution of aggregated IBD2/IBD0 log-likelihood ratios for each chromosome arm for self and non-self comparisons in the GBR, LWK, and Hair panel. In each panel, self comparisons are on top, non-self comparisons are on the bottom.

FIG. 12: Schematic of HiddenGem's score calculation and traceback algorithm.

### DETAILED DESCRIPTION

Before the methods, computer-readable media and systems of the present disclosure are described in greater detail, it is to be understood that the methods, computer-readable media and systems are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, computer-readable media and systems. The upper and lower limits of these smaller ranges may independently be included in the smaller ranges and are also encompassed within the methods, computer-readable media and systems, subject to any specifically excluded limit in the stated range. Where the stated range includes one or both of the limits, ranges excluding either or both of those included limits are also included in the methods, computer-readable media and systems.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, computer-readable media and systems belong. Although any methods, computer-readable media and systems similar or equivalent to those described herein can also be used in the practice or testing of the methods, computer-readable media and systems, representative illustrative methods, computer-readable media and systems are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, computer-readable media and systems are not entitled to antedate such publication, as the date of publication provided may be different from the actual publication date which may need to be independently confirmed. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, computer-readable media and systems, which are, for clarity, described in the context of separate embodiments, may also be provided in combination in a single embodiment. Conversely, various features of the methods, computer-readable media and systems, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, computer-readable media and systems and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Genetic Identification Methods

Aspects of the present disclosure include computer-implemented methods for comparing genotype data from a first sample to a limited amount of DNA sequence data from a second sample. Such methods are implemented using one or more processors and one or more non-transitory computer-readable media comprising instructions stored thereon, which when executed by the one or more processors, cause the one or more processors to perform operations. The operations comprise receiving genotype data from a first sample and a limited amount of DNA sequence data from a second sample, and comparing the genotype data and limited amount of DNA sequence data at a plurality of variable sites across one or more genomic regions. The operations further comprise, at each of the plurality of variable sites, calculating the likelihood of the limited amount of DNA sequence data and the genotype data being related under at least two models of relatedness, wherein the at least two models of relatedness comprise: (i) the first sample and the second sample share two chromosomes identical-by-descent (IBD2); and (ii) the first sample and the second sample share no chromosomes identical-by-descent (IBD0). As used herein, “IBD2”, “IBD1”, and “IBD0” mean that the samples being compared share 2, 1, and 0 chromosomes, respectively, of a chromosome pair as identical-by-descent. The operations further comprise, at each of the plurality of variable sites, comparing the likelihood of model (i) to the likelihood of model (ii). According to some embodiments, comparing the likelihood of model (i) to the likelihood of model (ii) comprises generating a log-likelihood ratio (LLR) of model (i) and model (ii), thereby generating a plurality of log-likelihood ratios comprising a log-likelihood ratio for each of the plurality of variable sites. In certain embodiments, such methods further comprise aggregating the plurality of log-likelihood ratios. Optionally, the log-likelihood ratios are aggregated across each arm of each autosome.

According to some embodiments, the first sample is from a known individual and the second sample is an unknown sample. By “unknown sample” is meant it is unknown whether the second sample is from the same individual as the first sample. Such methods may further comprise determining whether the unknown sample is from the known individual, e.g., based on aggregated log-likelihood ratios.

The methods of the present disclosure find use, and are advantageous in a variety of contexts. For example, several methods currently exist for detecting genetic relatedness by comparing DNA information. These methods generally require genotype calls, either SNPs or STRs, at the sites used for comparison. For some DNA samples, like those obtained from bone fragments or single rootless hairs, there is often not enough DNA present to generate genotype calls that are accurate and complete enough for these comparisons. The methods of the present disclosure, embodiments of which are sometimes referred to herein as “IBDGem”, constitute a fast and robust computational procedure for detecting genomic regions of identity-by-descent using low-coverage sequence data (e.g., low-coverage shotgun sequence data) and genotype calls from a known query individual. At less than one-fold genome coverage, IBDGem reliably detects segments of relatedness and can make high-confidence identity detections with as little as 1% genome coverage.

In certain embodiments, IBDGem does not attempt to call genotypes from the sequence data. Rather, IBDGem may be used to evaluate the likelihood that a test individual, whose genotype is known, could have generated the sequence data from the unknown (or “questioned”) sample. As demonstrated herein, this approach can reliably identify samples with as little as 0.01-fold genome coverage from the questioned sample. Consequently, among other useful applications, IBDGem enables forensic use of sample types such as bone and single rootless hairs that typically yield sub-nanogram quantities of fragmented DNA.

According to some embodiments, the methods are implemented in C and compare genotype data (e.g., generated via genotype array or DNA sequence data) from a known individual to aligned sequence data in BAM format from an unknown individual. In certain embodiments, for each variable site, it calculates the likelihood of the observed sequence data under 3 models of relatedness: (1) the compared samples share two chromosomes identical-by-descent, IBD2, (2) the compared samples share one chromosome identical-by-descent, IBD1, or (3) the compared samples share no chromosomes identical-by-descent, IBD0. These three relationships are the only possible ways that two samples can be related to one another at a particular region in the genome.

The likelihoods of the data under these three models can then be compared to find the most likely model or to generate a log-likelihood ratio. If the distances between variable sites are sufficiently large, i.e., longer than the length of a sequence read, then the observed alleles at each site can be treated as independent observations of the likelihood of each state (IBD2, IBD1, or IBD0) across a genomic region. Thus, these likelihoods can be aggregated across multiple sites to increase the discriminatory power across a genomic region.

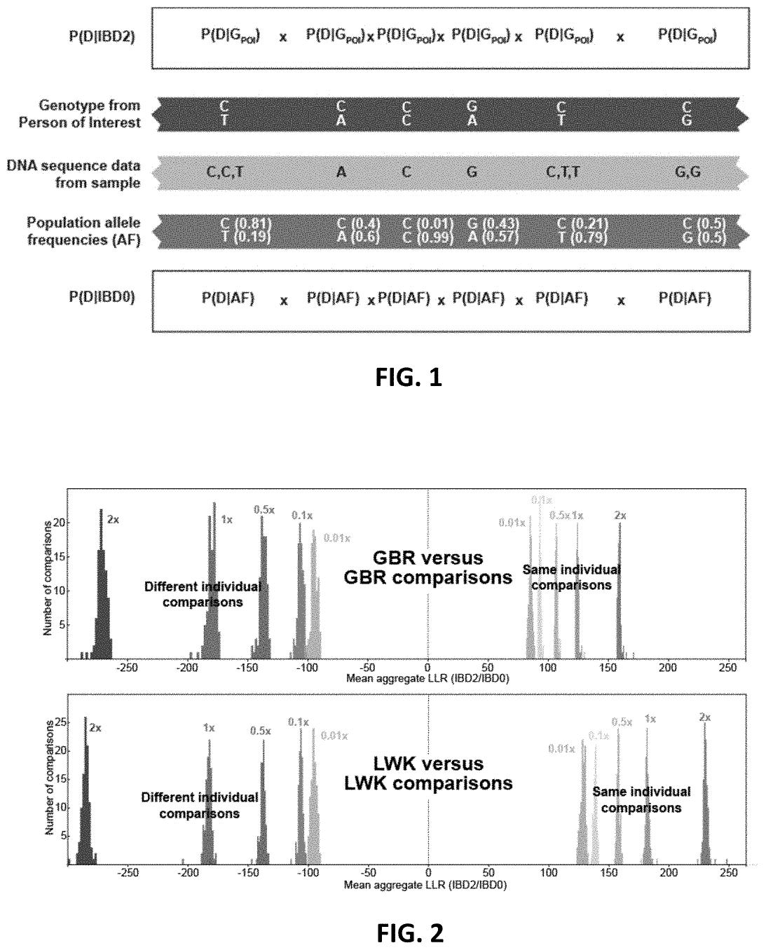

In the case of determining whether the sequence data derives from the same individual as the genotype data versus the model of it coming from an unrelated individual (analogous to the match probability for STR identification), log-odds ratios between the IBD2 and IBD0 models may be generated (FIG. 1). The calculations of likelihoods may be performed using the following non-limiting algorithmic approach.

Algorithmic Details for IBDGem The purpose of this program is to compare the data in an input BAM file (from shotgun sequencing of a hair, for example) against the variants in an input VCF file (from shotgun sequencing of a DNA sample or genotype array, for example). At each variant site in the input VCF file, likelihoods of the BAM data are generated under three models:
(1) that the BAM data are from an individual who has two identical-by-descent (IBD) chromosomes;
(2) that the BAM data are from an individual who has one IBD chromosome; and
(3) that the BAM data are from an individual who has zero IBD chromosomes

IBDGem requires population allele frequency estimates for each variant site. This information can come from an AF tag in an input VCF file. This information is used in the likelihood calculation for non-IBD chromosomes.

To formalize the procedure, let us define data for a given variable site in the genome, Sᵢ, that is present in the VCF file, and for which there is observed data in an input BAM file.

Let Gᵢ represent the known genotype of Sᵢ in the VCF file with 0 representing the reference allele and 1 representing the alternative allele. The alleles are not in any order, i.e., are unphased. Only bi-allelic sites are considered.

Gᵢ=(0|1,0|1)

Note that Gᵢ must take one of three possible values: (0,0), (0,1), or (1,1).

Let Dᵢ represent the observed data from a BAM file, e.g., of observed reference and alternative allele counts at site Sᵢ

Dᵢ=(Dᵢ₀,Dᵢ₁)

The probability of the observed data, Di, under each of the three possible genotypes of the data in the BAM data sample can be defined:

$$P ( {D}_{i} \vert G ) = \{ \begin{matrix}{( 1 - ε )}^{{D}_{i 0}} {( ε )}^{{D}_{i 1}} , & G = ( 0 , 0 ) \\ {( 1 - ε )}^{{D}_{i 1}} {( ε )}^{{D}_{i 0}} , & G = ( 1 , 1 ) \\ ( \begin{matrix}{D}_{i o} + {D}_{i 1} \\ {D}_{i o}\end{matrix} ) {( \frac{1}{2} )}^{{D}_{i o}} {( \frac{1}{2} )}^{{D}_{i 1}} , & G = ( 0 , 1 )\end{matrix} \qquad Equation 1$$

Where ε is the probability of wrongly observed an allele, i.e., sequencing error. Note that these probabilities for homozygous genotypes are simply the probability of correctly observing the only allele present in the data the number of times it was observed given the sequencing error rate. For the heterozygous case, the probability of the data is the binomial probability of the data given the number of each of the two alleles observed. Further, it is assumed that errors are symmetric, such that observing an alternative allele in a read when the reference allele was present occurs as often as observing a reference allele in a read when the alternative allele was present.

With these probabilities of data, given an underlying genotype, the probability of observed data can be calculated given an allele's frequency:

P(Dᵢ|f)=(1−f)²P(Dᵢ|Gᵢ=(0,0))+2f(1−f)P(Dᵢ|Gᵢ=(0,1))+f²P(Dᵢ|Gᵢ=(1,1)) Equation 2

This equation assumes Hardy-Weinberg equilibrium. It represents the probability of BAM data given no information from the VCF file, i.e., only the frequency of the allele in the population. Thus, this represents a model of the probability of data under the assumption that the comparison VCF file shares zero IBD chromosomes with the BAM file individual.

The probability of the BAM data, Dᵢ, at a particular variable site of known alternative allele frequency, f, can be calculated thusly:

P(Dᵢ|Gᵢ,f,IBD0)=P(Dᵢ|f)

That is, the probability of the BAM data, given that it comes from a sample that is IBD0 with the VCF data is simply determined by the frequency, f, of the alternative allele as described in Equation 2.

P(Dᵢ|Gᵢ,f,IBD2)=P(Dᵢ|Gᵢ)

That is, the probability of the BAM data, given that it comes from a sample that is IBD2 with the VCF data is simply determined by the genotype of the VCF file, as described in Equation 1.

The IBD1 calculation (unlike the IBD0 and IBD2 calculations), is calculated differently depending on the genotype of the VCF file, G_{L}. Under this model, the BAM data individual shares one allele with the VCF individual. The other is not shared. What can be assumed about the shared allele depends on whether the VCF individual is homozygous or heterozygous. IBD1 probabilities are calculated thusly:

$$P ( {D}_{i} \vert G , f , IBD 1 ) = \{ \begin{matrix}f ( \begin{matrix}{D}_{i o} + {D}_{i 1} \\ {D}_{i o}\end{matrix} ) {( \frac{1}{2} )}^{{D}_{i o}} {( \frac{1}{2} )}^{{D}_{i 1}} + ( 1 - f ) {( 1 - ε )}^{{D}_{i 0}} {( ε )}^{{D}_{i 1}} , & G = ( 0 , 0 ) \\ ( 1 - f ) ( \begin{matrix}{D}_{i o} + {D}_{i 1} \\ {D}_{i o}\end{matrix} ) {( \frac{1}{2} )}^{{D}_{i o}} {( \frac{1}{2} )}^{{D}_{i 1}} + {f ( ε )}^{{D}_{i 0}} {( 1 - ε )}^{{D}_{i 1}} , & G = ( 1 , 1 ) \\ ( \frac{1 - f}{2} ) ( {( 1 - ε )}^{{D}_{i 0}} {( ε )}^{{D}_{i 1}} ) + ( \frac{f}{2} ) ( {( 1 - ε )}^{{D}_{i 1}} {( ε )}^{{D}_{i 0}} ) + ( \frac{1}{2} ) ( ( \begin{matrix}{D}_{i o} + {D}_{i 1} \\ {D}_{i o}\end{matrix} ) {( \frac{1}{2} )}^{{D}_{i o}} {( \frac{1}{2} )}^{{D}_{i 1}} ) , & G = ( 0 , 1 )\end{matrix} \qquad Equation 3$$

The rationale for these equations is that the probability of the underlying genotypes of the BAM data sample can be calculated knowing the genotype of the VCF data and the frequency of the derived allele at that site. Then, the probability of the BAM data, given a particular comparison genotype can be calculated.

The log-odds ratio (LOR) of any two of these models (IBD0, IBD1, and IBD2) can then be calculated for the data at a given site. Because data are independent across sites, one can aggregate the LOR within bins to increase power to discriminate between models. The case of distinguishing between whether the two samples are from the same individual and whether they are from different, unrelated individuals is a comparison of the IBD2 versus IBD0 model across all arms of all chromosomes.

To analyze signals that are regional over the genome, the genome may be divided into non-overlapping bins of sufficient length such that each bin will contain tens or hundreds of sites. Then, the aggregate LOR in each bin over the genome is calculated.

According to some embodiments, the methods of the present disclosure are computer-implemented. As used herein, “computer-implemented” means at least one step of the method is implemented using one or more processors and one or more non-transitory computer-readable media. The computer-implemented methods of the present disclosure may further comprise one or more steps that are not computer-implemented, e.g., obtaining one or more samples (e.g., a forensic sample), preparing the one or more samples for genotyping and/or nucleic acid sequencing, and/or the like.

In certain embodiments, receiving the genotype data comprises receiving a VCF file comprising the genotype data. According to some embodiments, the genotype data was generated by massively parallel sequencing (MPS). Sequencing may be performed using any of a variety of available MPS sequencing machines and systems. Illustrative sequencing systems include the Illumina iSeq 100, Miniseq, MiSeq series, NextSeq series (e.g., NextSeq 500 series, NextSeq 1000, NextSeq 2000), and NovaSeq sequencing systems (Illumina, Inc., San Diego, Calif.), the Pacific Biosciences Sequel (e.g., Sequel II) sequencing system (Pacific Biosciences, Menlo Park, Calif.), the Oxford Nanopore Technologies MinION™, GridIONx5™, PromethION™, or SmidgION™ nanopore-based sequencing systems (Oxford Nanopore Technologies, Oxford, UK), and other systems having similar capabilities.

According to some embodiments, the genotype data was generated by genotype array. Suitable genotype array technologies are known and include, but are not limited to, the Illumina Mutli-Ethnic Global Screening Array (MEGA) and the Illumina Global Screening Array (GSA).

In certain embodiments, receiving the sequence data comprises receiving a BAM file comprising the sequence data.

According to some embodiments, the variable sites comprise single-nucleotide polymorphisms (SNPs), insertion-deletions (INDELs), or a combination thereof.

In certain embodiments, the unknown sample is a hair sample, a bone sample, a blood sample, a semen sample, or any combination thereof. When the unknown sample comprises a hair sample (e.g., a head hair sample, a pubic hair sample, or the like), in some instances, the hair sample is a rootless hair sample. For example, according to some embodiments, the unknown sample comprises a single rootless hair sample.

As will be appreciated with the benefit of the present disclosure, the methods of the present disclosure find use in performing a forensic analysis. In certain embodiments, the known individual is a person of interest (P01) in a criminal investigation, e.g., a murder investigation, a rape investigation, and/or the like. Accordingly, in some instances, the unknown sample was collected from a crime scene or a victim of a crime, e.g., a murder victim or a rape victim.

In certain embodiments, the limited amount of DNA sequence data comprises less than 1-fold genome coverage, less than 0.5-fold genome coverage, less than 0.1-fold genome coverage, or less than 0.05-fold genome coverage. According to some embodiments, the limited amount of DNA sequence data was obtained from a sample comprising less than 1 nanogram of genomic DNA.

### Relatedness Detection Methods

Aspects of the present disclosure also include computer-implemented methods for assessing the degree of relatedness between genotype data from a first sample and a limited amount of DNA sequence data from a second sample. Such methods are implemented using one or more processors and one or more non-transitory computer-readable media comprising instructions stored thereon, which when executed by the one or more processors, cause the one or more processors to perform operations. The operations comprise receiving genotype data from a first sample from a first individual and a limited amount of DNA sequence data from a second sample from a second individual, and comparing the genotype data and limited amount of DNA sequence data at a plurality of variable sites across one or more genomic regions. In some embodiments, the second sample from the second individual is a hair sample (e.g., a root-containing hair, a rootless hair, or the like) from the second individual. The operations further comprise, at each of the plurality of variable sites, calculating the likelihood of the limited amount of DNA sequence data and the genotype data being related under the following models of relatedness: (i) the first sample and the second sample share two chromosomes identical-by-descent (IBD2); (ii) the first sample and the second sample share one chromosome identical-by-descent (IBD1); and (iii) the first sample and the second sample share no chromosomes identical-by-descent (IBD0). The operations further comprise determining the most likely path of the three IBD models through the one or more genomic regions using regional likelihood values of each model. In some instances, the genotype data from the first sample is available from an online database.

According to some embodiments, determining the most likely path of the three IBD states is performed using a dynamic programming algorithm. Non-limiting examples of suitable dynamic programming algorithms which may be employed include a forward algorithm, a forward-backward algorithm, or a Viterbi algorithm.

In certain embodiments, the operations further comprise partitioning the most likely path into IBD2, IBD1 and IBD0. In some instances, the operations further comprise identifying regions of co-inheritance for IBD2, IBD1 and/or IBD0. According to some embodiments, two individuals (e.g., cousins) are related to the second individual (e.g., a grandmother), and wherein the operations further comprise identifying IBD1 segments that are shared between the two individuals but not shared between the two individuals and the second individual, thereby identifying IBD1 segments from a common ancestor (e.g., a grandfather) of the two individuals other than the second individual.

According to some embodiments, the methods for assessing the degree of relatedness of the present disclosure further comprising, based on the determining step, identifying the first and second individuals as parent-child relatives. In certain embodiments, the methods for assessing the degree of relatedness of the present disclosure further comprising, based on the determining step, identifying the first and second individuals as full siblings.

Algorithmic details for an embodiment (sometimes referred to herein as “HiddenGem”) of the methods for assessing the degree of relatedness of the present disclosure will now be described.

Algorithmic Details for HiddenGem

The HiddenGem module employs a maximum-likelihood approach to find the most likely path of IBD states across a genomic region using likelihood values calculated by the main program for each IBD model.

Following comparison of two samples by IBDGem, likelihood values are aggregated into non-overlapping bins containing a fixed number of sites, for a total of N bins. Then, at the b-th bin along the genomic region, the likelihoods for models IBD0, IBD1, and IBD2 are first normalized to probabilities as follows:

$$P ( {M}_{b} ) = \frac{L ( {M}_{b} )}{L ( IBD {0}_{b} ) + L ( IBD {1}_{b} ) + L ( IBD {2}_{b} )} ( b \leq N )$$

where Me∈(IBD0,IBD1,IBD2).

Moving from the first to last bin, for each of the 3 IBD states, a 3× N score matrix is populated by multiplying the cumulative probability of state M_{b-1} in the previous bin with the probability of state M_{b} in the current bin and a penalty for switching states if M_{b-1}≠M_{b}, keeping the largest product as the current score at M_{b}. The score calculation for IBD0, IBD1, and IBD2 at bin b can thus be formalized as follows:

$$Score ( IBD {0}_{b} ) = max \{ \begin{matrix}Score ( IBD {0}_{b - 1} ) P ( IBD {0}_{b} ) , \\ Score ( IBD {1}_{b - 1} ) P ( IBD {0}_{b} ) {w}_{IBD 0 - IBD 1} , \\ Score ( IBD {2}_{b - 1} ) P ( IBD {0}_{b} ) {w}_{IBD 0 - IBD 2}\end{matrix}$$

$$Score ( IBD {1}_{b} ) = max \{ \begin{matrix}Score ( IBD {0}_{b - 1} ) P ( IBD {1}_{b} ) {w}_{IBD 0 - IBD 1} , \\ Score ( IBD {1}_{b - 1} ) P ( IBD {1}_{b} ) , \\ Score ( IBD {2}_{b - 1} ) P ( IBD {1}_{b} ) {w}_{IBD 1 - IBD 2}\end{matrix}$$

$$Score ( IBD {2}_{b} ) = max \{ \begin{matrix}Score ( IBD {0}_{b - 1} ) P ( IBD {2}_{b} ) {w}_{IBD 0 - IBD 1} , \\ Score ( IBD {1}_{b - 1} ) P ( IBD {2}_{b} ) {w}_{IBD 1 - IBD 2} , \\ Score ( IBD {2}_{b - 1} ) P ( IBD {2}_{b} )\end{matrix}$$

where w_{IBD0-IBD1} is the switch penalty between IBD0 and IBD1, w_{IBD0-IBD2} is the switch penalty between IBD0 and IBD2, and w_{IBD1-IBD2} is the switch penalty between IBD1 and IBD2.

The score matrix also keeps track of which state in the previous bin b−1 yields the highest score in the current bin b, so that when scores for the last bin have been calculated, backtracking along the matrix is performed to find the path of IBD states that results in the final maximum cumulative probability.

The path of states provided by HiddenGem can then be used to estimate the proportion of each IBD state across the genome, for example by counting the number of bins at a specific state, and infer the degree of relatedness between the compared samples.

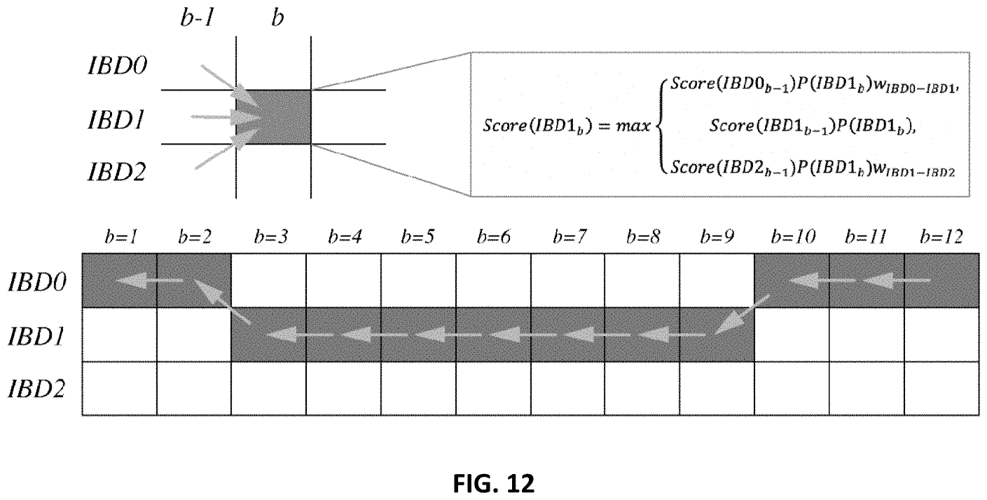

Shown in FIG. 12 is a schematic of HiddenGem's score calculation and traceback algorithm.

### Computer-Readable Media and Systems

Aspects of the present disclosure further include systems and non-transitory computer-readable media. In certain aspects, provided are one or more computer-readable media comprising instructions stored thereon, which when executed by one or more processors, cause the one or more processors to perform the operations of any of the computer-implemented methods of the present disclosure.

In other aspects, provided are systems comprising one or more processors and one or more computer-readable media comprising instructions stored thereon, which when executed by the one or more processors, cause the one or more processors to perform the operations of any of the computer-implemented methods of the present disclosure.

A variety of processor-based systems may be employed to implement the embodiments of the present disclosure. Such systems may include system architecture wherein the components of the system are in electrical communication with each other using a bus. System architecture can include a processing unit (CPU or processor), as well as a cache, that are variously coupled to the system bus. The bus couples various system components including system memory, (e.g., read only memory (ROM) and random access memory (RAM), to the processor.

System architecture can include a cache of high-speed memory connected directly with, in close proximity to, or integrated as part of the processor. System architecture can copy data from the memory and/or the storage device to the cache for quick access by the processor. In this way, the cache can provide a performance boost that avoids processor delays while waiting for data. These and other modules can control or be configured to control the processor to perform various actions. Other system memory may be available for use as well. Memory can include multiple different types of memory with different performance characteristics. Processor can include any general purpose processor and a hardware module or software module, such as first, second and third modules stored in the storage device, configured to control the processor as well as a special-purpose processor where software instructions are incorporated into the actual processor design. The processor may essentially be a completely self-contained computing system, containing multiple cores or processors, a bus, memory controller, cache, etc. A multi-core processor may be symmetric or asymmetric.

To enable user interaction with the computing system architecture, an input device can represent any number of input mechanisms, such as a microphone for speech, a touch-sensitive screen for gesture or graphical input, keyboard, mouse, motion input, speech and so forth. An output device can also be one or more of a number of output mechanisms. In some instances, multimodal systems can enable a user to provide multiple types of input to communicate with the computing system architecture. A communications interface can generally govern and manage the user input and system output. There is no restriction on operating on any particular hardware arrangement and therefore the basic features here may easily be substituted for improved hardware or firmware arrangements as they are developed.

The storage device is typically a non-volatile memory and can be a hard disk or other types of computer-readable media which can store data that are accessible by a computer, such as magnetic cassettes, flash memory cards, solid state memory devices, digital versatile disks, cartridges, random access memories (RAMs), read only memory (ROM), and hybrids thereof.

The storage device can include software modules for controlling the processor. Other hardware or software modules are contemplated. The storage device can be connected to the system bus. In one aspect, a hardware module that performs a particular function can include the software component stored in a computer-readable medium in connection with the necessary hardware components, such as the processor, bus, output device, and so forth, to carry out various functions of the disclosed technology.

Embodiments within the scope of the present disclosure may also include tangible and/or non-transitory computer-readable storage media or devices for carrying or having computer-executable instructions or data structures stored thereon. Such tangible computer-readable storage devices can be any available device that can be accessed by a general purpose or special purpose computer, including the functional design of any special purpose processor as described above. By way of example, and not limitation, such tangible computer-readable devices can include RAM, ROM, EEPROM, CD-ROM or other optical disk storage, magnetic disk storage or other magnetic storage devices, or any other device which can be used to carry or store desired program code in the form of computer-executable instructions, data structures, or processor chip design. When information or instructions are provided via a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform tasks or implement abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

Notwithstanding the appended claims, the present disclosure is also defined by the following embodiments.

1. A computer-implemented method for comparing genotype data from a first sample to a limited amount of DNA sequence data from a second sample, the method being implemented using one or more processors and one or more non-transitory computer-readable media comprising instructions stored thereon, which when executed by the one or more processors, cause the one or more processors to perform operations comprising:
(a) receiving genotype data from a first sample and a limited amount of DNA sequence data from a second sample;
(b) comparing the genotype data and limited amount of DNA sequence data at a plurality of variable sites across one or more genomic regions;
(c) at each of the plurality of variable sites, calculating the likelihood of the limited amount of DNA sequence data and the genotype data being related under at least two models of relatedness, wherein the at least two models of relatedness comprise:
   (i) the first sample and the second sample share two chromosomes identical-by-descent (IBD2); and
   (ii) the first sample and the second sample share no chromosomes identical-by-descent (IBD0);
(d) at each of the plurality of variable sites, comparing the likelihood of model (i) to the likelihood of model (ii).
   2. The computer-implemented method according to embodiment 1, wherein comparing the likelihood of model (i) to the likelihood of model (ii) comprises generating a log-likelihood ratio of model (i) and model (ii), thereby generating a plurality of log-likelihood ratios comprising a log-likelihood ratio for each of the plurality of variable sites.
   3. The computer-implemented method according to embodiment 2, further comprising aggregating the plurality of log-likelihood ratios.
   4. The computer-implemented method according to embodiment 3, wherein log-likelihood ratios are aggregated across each arm of each autosome.
   5. The computer-implemented method according to any one of embodiments 1 to 4, wherein the first sample is from a known individual and the second sample is an unknown sample.
   6. The computer-implemented method according to embodiment 5, further comprising determining whether the unknown sample is from the known individual.
   7. The computer-implemented method according to embodiment 6, wherein determining whether the unknown sample is from the known individual is based on the aggregated log-likelihood ratios of embodiment 3 or embodiment 4.
   8. The computer-implemented method according to any one of embodiments 1 to 7, wherein receiving the genotype data comprises receiving a VCF file comprising the genotype data.
   9. The computer-implemented method according to any one of embodiments 1 to 8, wherein the genotype data was generated by massively parallel sequencing (MPS).
   10. The computer-implemented method according to any one of embodiments 1 to 8, wherein the genotype data was generated by genotype array.
   11. The computer-implemented method according to any one of embodiments 1 to 10, wherein receiving the sequence data comprises receiving a BAM file comprising the sequence data.
   12. The computer-implemented method according to any one of embodiments 1 to 11, wherein the variable sites comprise single-nucleotide polymorphisms (SNPs), insertion-deletions (INDELs), or a combination thereof.
   13. The computer-implemented method according to any one of embodiments 1 to 11, wherein the second sample is a hair sample, a bone sample, a blood sample, a semen sample, or any combination thereof.
   14. The computer-implemented method according to embodiment 13, wherein the hair sample is a rootless hair sample.
   15. The computer-implemented method according to embodiment 14, wherein the hair sample is a single rootless hair sample.
   16. The computer-implemented method according to any one of embodiments 1 to 15, wherein the second sample was collected from a crime scene.
   17. The computer-implemented method according to any one of embodiments 1 to 16, wherein the first sample is from a person of interest in a criminal investigation.
   18. The computer-implemented method according to embodiment 16 or embodiment 17, wherein the method is performed for a forensic analysis.
   19. The computer-implemented method according to any one of embodiments 1 to 18, wherein the limited amount of DNA sequence data comprises less than 2-fold genome coverage, less than 1.5-fold genome coverage, less than 1-fold genome coverage, less than 0.5-fold genome coverage, less than 0.1-fold genome coverage, or less than 0.05-fold genome coverage.
   20. The computer-implemented method according to any one of embodiments 1 to 19, wherein the limited amount of DNA sequence data was obtained from a sample comprising less than 1 nanogram of genomic DNA.
   21. One or more non-transitory computer-readable media comprising instructions stored thereon, which when executed by one or more processors, cause the one or more processors to perform operations comprising:
(a) receiving genotype data from a first sample and a limited amount of DNA sequence data from a second sample;
(b) comparing the genotype data and limited amount of DNA sequence data at a plurality of variable sites across one or more genomic regions;
(c) at each of the plurality of variable sites, calculating the likelihood of the limited amount of DNA sequence data and the genotype data being related under at least two models of relatedness, wherein the at least two models of relatedness comprise:
   (i) the first sample and the second sample share two chromosomes identical-by-descent (IBD2); and
   (ii) the first sample and the second sample share no chromosomes identical-by-descent (IBD0);
(d) at each of the plurality of variable sites, comparing the likelihood of model (i) to the likelihood of model (ii).
   22. The one or more non-transitory computer-readable media of embodiment 21, wherein comparing the likelihood of model (i) to the likelihood of model (ii) comprises generating a log-likelihood ratio of model (i) and model (ii), thereby generating a plurality of log-likelihood ratios comprising a log-likelihood ratio for each of the plurality of variable sites.
   23. The one or more non-transitory computer-readable media of embodiment 22, wherein the operations further comprise aggregating the plurality of log-likelihood ratios.
   24. The one or more non-transitory computer-readable media of embodiment 23, wherein log-likelihood ratios are aggregated across each arm of each autosome.
   25. The one or more non-transitory computer-readable media of any one of embodiments 21 to 24, wherein the first sample is from a known individual and the second sample is an unknown sample.
   26. The one or more non-transitory computer-readable media of embodiment 25, wherein the operations further comprise determining whether the unknown sample is from the known individual based on the aggregated log-likelihood ratios.
   27. One or more non-transitory computer-readable media comprising instructions stored thereon, which when executed by one or more processors, cause the one or more processors to perform the computer-implemented method according to any one of embodiments 1 to 20.
   28. A computer system comprising the one or more non-transitory computer-readable media of any one of embodiments 21 to 27.
   29. A computer-implemented method for assessing the degree of relatedness between genotype data from a first sample and a limited amount of DNA sequence data from a second sample, the method being implemented using one or more processors and one or more non-transitory computer-readable media comprising instructions stored thereon, which when executed by the one or more processors, cause the one or more processors to perform operations comprising:
(a) receiving genotype data from a first sample from a first individual and a limited amount of DNA sequence data from a second sample from a second individual;
(b) comparing the genotype data and limited amount of DNA sequence data at a plurality of variable sites across one or more genomic regions;
(c) at each of the plurality of variable sites, calculating the likelihood of the limited amount of DNA sequence data and the genotype data being related under the following models of relatedness:
   (i) the first sample and the second sample share two chromosomes identical-by-descent (IBD2);
   (ii) the first sample and the second sample share one chromosome identical-by-descent (IBD1); and
   (iii) the first sample and the second sample share no chromosomes identical-by-descent (IBD0); and
(d) determining the most likely path of the three IBD models through the one or more genomic regions using regional likelihood values of each model.
   30. The computer-implemented method according to embodiment 29, wherein the genotype data from the first sample is available from an online database.
   31. The computer-implemented method according to embodiment 29 or embodiment 30, wherein step (d) is performed using a dynamic programming algorithm.
   32. The computer-implemented method according to embodiment 31, wherein the dynamic programming algorithm comprises a forward algorithm, a forward-backward algorithm, or a Viterbi algorithm.
   33. The computer-implemented method according to any one of embodiments 29 to 32, wherein the operations further comprise partitioning the most likely path into IBD2, IBD1 and IBD0.
   34. The computer-implemented method according to any one of embodiments 29 to 33, wherein the operations further comprise identifying regions of co-inheritance for IBD2, IBD1 and/or IBD0.
   35. The computer-implemented method according to any one of embodiments 29 to 34, wherein two individuals are related to the second individual, and wherein the operations further comprise identifying IBD1 segments that are shared between the two individuals but not shared between the two individuals and the second individual, thereby identifying IBD1 segments from a common ancestor of the two individuals other than the second individual.
   36. The computer-implemented method according to any one of embodiments 29 to 35, further comprising, based on the determination at step (d), identifying the first and second individuals as parent-child relatives.
   37. The computer-implemented method according to any one of embodiments 29 to 35, further comprising, based on the determination at step (d), identifying the first and second individuals as full siblings.
   38. One or more non-transitory computer-readable media comprising instructions stored thereon, which when executed by one or more processors, cause the one or more processors to perform operations comprising:
(a) receiving genotype data from a first sample from a first individual and a limited amount of DNA sequence data from a second sample from a second individual;
(b) comparing the genotype data and limited amount of DNA sequence data at a plurality of variable sites across one or more genomic regions;
(c) at each of the plurality of variable sites, calculating the likelihood of the limited amount of DNA sequence data and the genotype data being related under the following models of relatedness:
   (i) the first sample and the second sample share two chromosomes identical-by-descent (IBD2);
   (ii) the first sample and the second sample share one chromosome identical-by-descent (IBD1); and
   (iii) the first sample and the second sample share no chromosomes identical-by-descent (IBD0); and
(d) determining the most likely path of the three IBD states through the one or more genomic regions using regional likelihood values of each model.
   39. One or more non-transitory computer-readable media comprising instructions stored thereon, which when executed by one or more processors, cause the one or more processors to perform the computer-implemented method according to any one of embodiments 29 to 37.
   40. A computer system comprising the one or more non-transitory computer-readable media of embodiment 39.

The following examples are offered by way of illustration and not by way of limitation.

### EXPERIMENTAL

### Example 1—IBDGem Sample Comparison

IBDGem analyzes regions of the genome for which there is genotype data from a known sample and some amount of sequence data from an unknown sample. It calculates the likelihood of the two samples being related by 0, 1, or 2 shared chromosomes (identical-by-descent) regionally across the genome. Because humans are diploids, humans carry two copies of each genomic locus. Thus, these three models (IBD0, IBD1, and IBD2) are the only ways that two samples can be related at any particular genomic region. More closely related individuals have more IBD1 regions (genome segments inherited from common ancestors) than less closely related individuals.

Comparisons between unrelated individuals will be IBD0, i.e., not share either chromosomal region from a recent common ancestor, across all or nearly all regions of the genome. Conversely, comparisons between the same person will necessarily be IBD2 across every region of the genome. For closely related individuals, some regions will be IBD1, where a segment of a chromosome is co-inherited from a recent common ancestor. Parent-offspring relatives are IBD1 across all chromosomes. Full siblings are roughly 25% IBD0, 50% IBD1, and 25% IBD2.

To test the ability of IBDGem to reliably compare samples, data from the high-coverage 1000 Genomes panel [15] was first used. This panel provides both genotype calls and aligned DNA sequence data for each individual. In this analysis, self versus self comparisons represent positive controls wherein all segments of all chromosomes should be identifiable as IBD2. Further, self versus non-self comparisons represent negative controls wherein all segments of all chromosomes should be identifiable as IBD0, except in cases of cryptic relatedness, which is known to be present in this panel.

The genotype and sequence data in the GBR (British) and LWK (Luhya) panels from the 1000 Genomes was first analyzed. All available genotypes at bi-allelic SNP sites was used for these comparisons. In this way, this experiment approximates the situation of having high-coverage DNA from one comparison individual with which to generate full genotype information. After excluding known relatives, one self and one non-self comparison was performed for every sample within each panel. Specifically, the genotype of each individual was compared against either their own aligned sequence data (self comparison) or the sequence data of a different, random individual within the same panel (non-self comparison) that had been down-sampled to 2-fold, 1-fold, 0.5-fold, 0.1-fold, and 0.01-fold genomic coverages. For the background model (IBD0), allele-frequencies from all unrelated individuals in the 1000 Genomes panel were used.

Variance in mean aggregated LLR across GBR and LWK individuals for self and non-self comparisons, at different down-sampling coverages.

| | Self comparison | | | | | Non-self comparison | | | | |
|---|---|---|---|---|---|---|---|---|---|---|
| | 2x | 1x | 0.5x | 0.1x | 0.01x | 2x | 1x | 0.5x | 0.1x | 0.01x |
| GBR | 3.330 | 1.577 | 1.034 | 1.008 | 2.125 | 30.83 | 14.74 | 8.677 | 6.073 | 7.687 |
| LWK | 57.81 | 20.19 | 5.341 | 1.568 | 3.988 | 446.2 | 117.8 | 24.20 | 3.820 | 5.430 |

Variance in mean aggregated LLR across GBR and LWK individuals for self and non-self comparisons, using different populations as background model.

| | | | | | | LWK (for |
|---|---|---|---|---|---|---|
| | | | | | | GBR)/GBR |
| | AFR | AMR | EAS | EUR | SAS | (for LWK) |
| | Self comparison | | | | | |
| GBR | 2.843 | 1.950 | 3.314 | 1.655 | 1.880 | 3.244 |
| LWK | 17.57 | 40.77 | 93.08 | 70.26 | 73.66 | 88.62 |
| | Non-self comparison | | | | | |
| GBR | 14.94 | 14.24 | 13.69 | 14.36 | 14.33 | 14.87 |
| LWK | 113.6 | 101.7 | 78.94 | 90.71 | 89.70 | 82.13 |

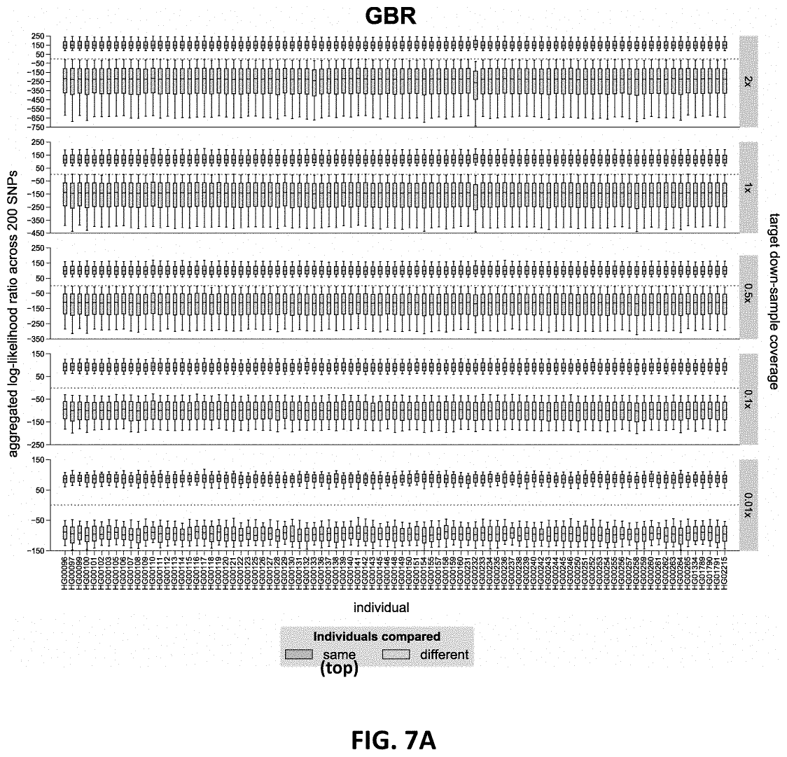

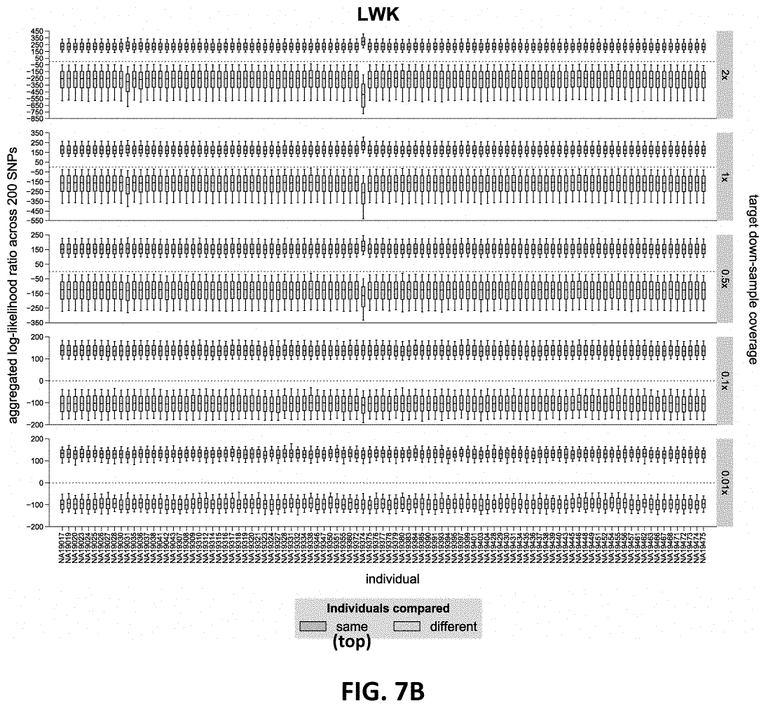

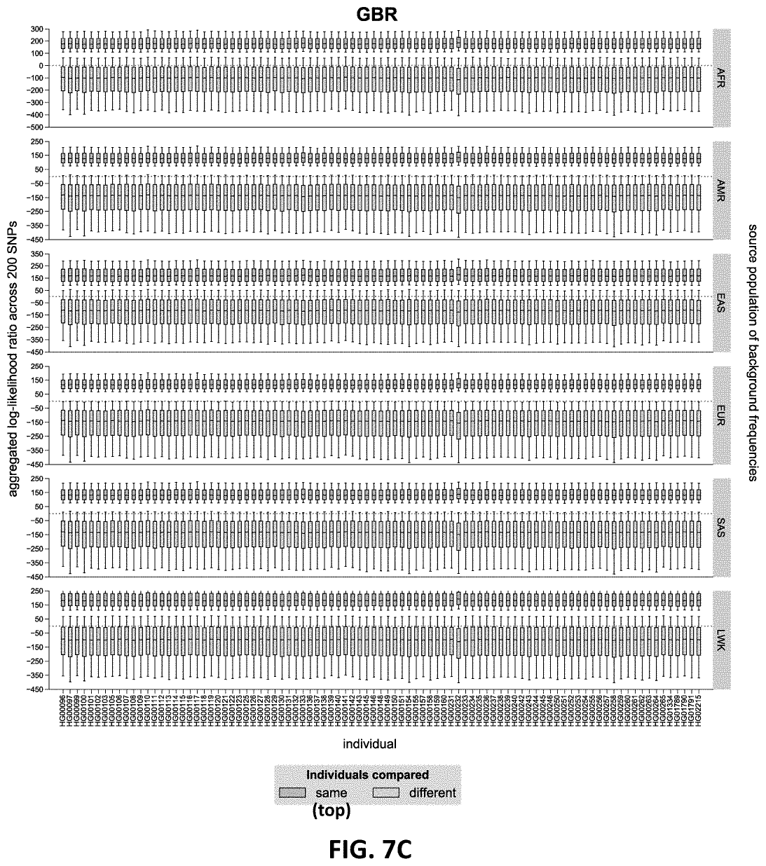

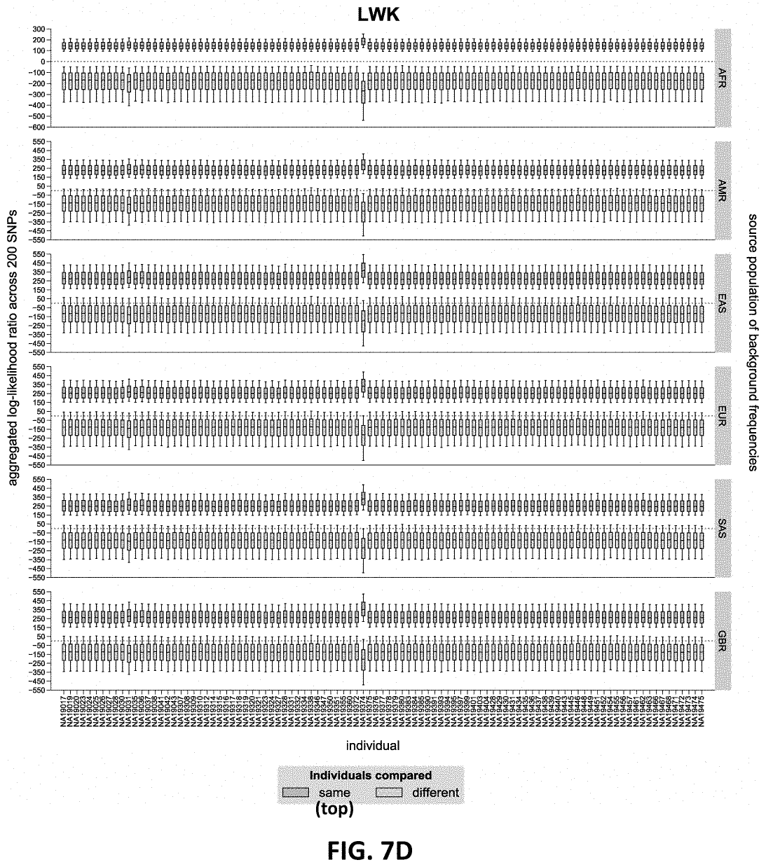

For all of these comparisons, it was found that self comparisons were strongly identifiable from non-self comparisons, even at the ultra-low coverage of 0.01-fold (FIG. 2 and FIG. 7A-7B).

Because the probability models for IBD0 and IBD1 take as input the non-reference allele frequency at each variable site, the sensitivity of IBDGem to these input values was tested. Human populations, in general, have low cross-population Fₛₜ values [17]. Thus, a priori IBDGem is not expected to be sensitive to the small differences in allele frequencies from various human populations.

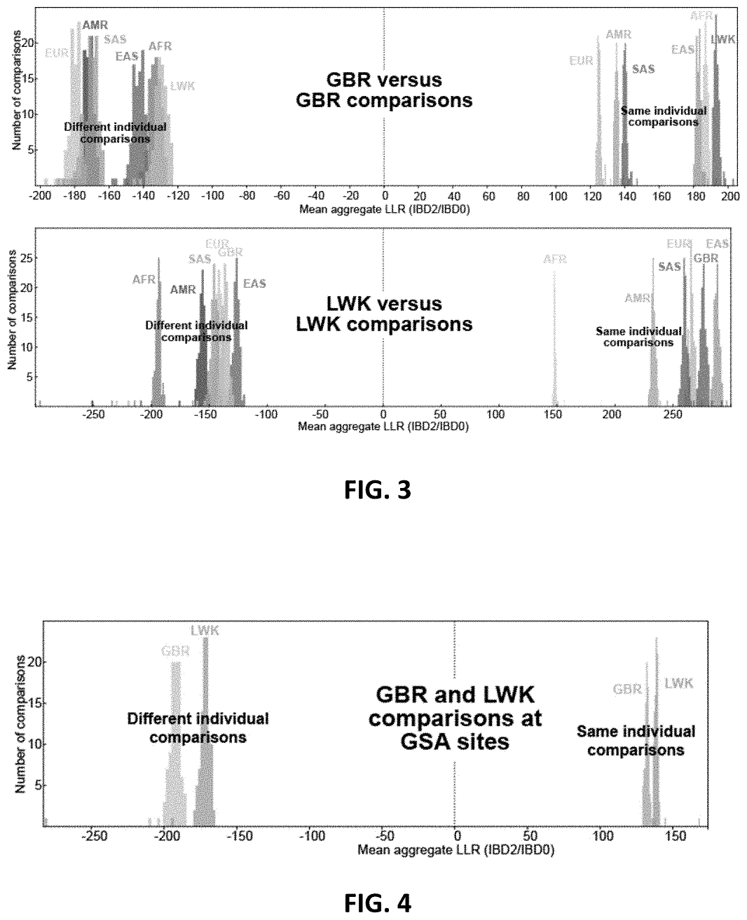

To test this expectation, the previous comparisons of 1-fold genome coverage data from the GBR and LWK panels were re-ran. For this experiment, the most general model of allele frequencies, i.e., derived from all unrelated individuals from the 1000 Genomes panel, were not used. Instead, allele frequencies from specific continental or sub-continental subsets were used (FIG. 3 and FIG. 7C-7D). In each case, the comparisons strongly identified data from the same individual, regardless of the population used to model background allele frequencies. For example, the genotypes of all GBR individuals were identifiable from 1-fold random genome coverage with log-likelihood ratio means of greater than 100 across genomic bins even when the background allele frequency models were learned from the SAS (South Asian) or AFR (African) superpopulations in the 1000 Genomes data. Non-self comparisons were similarly identifiable as such, despite using a population to which the individual does not belong to model background allele frequencies.

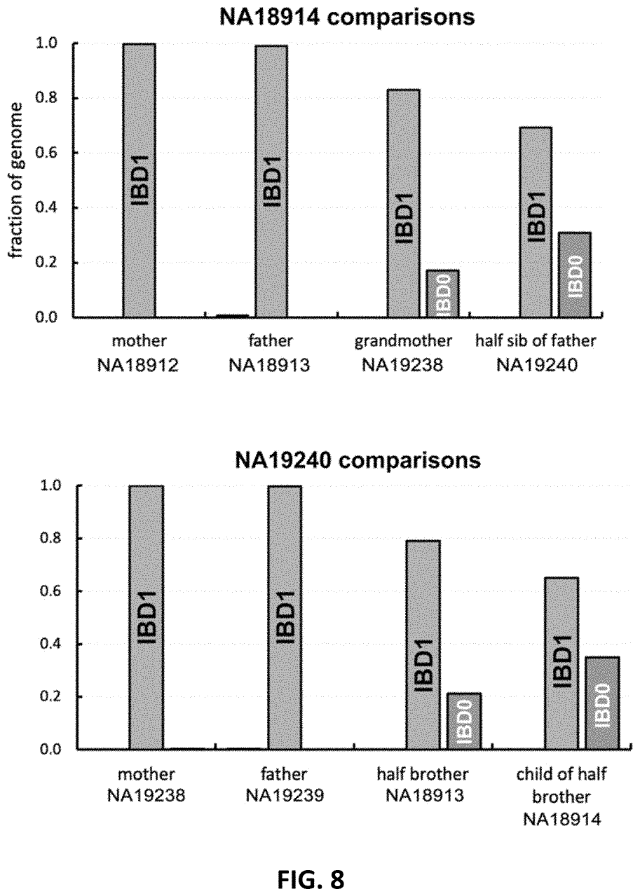

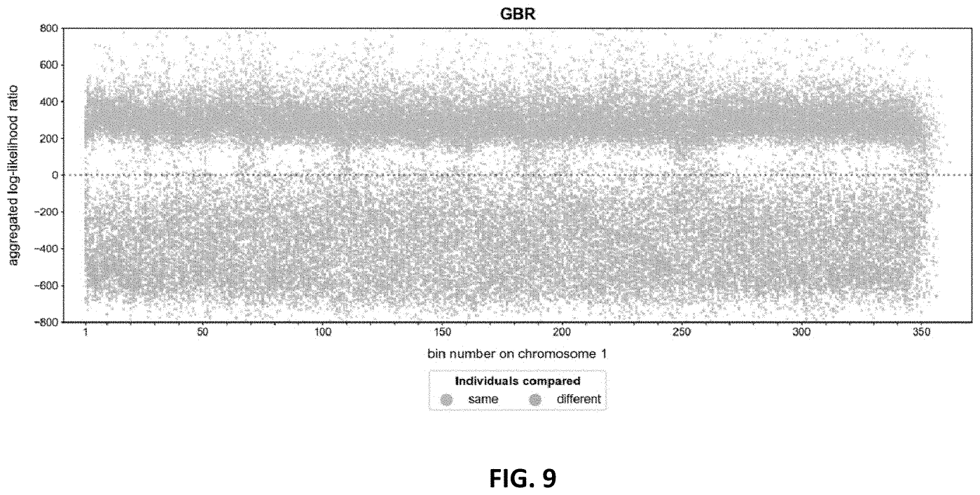

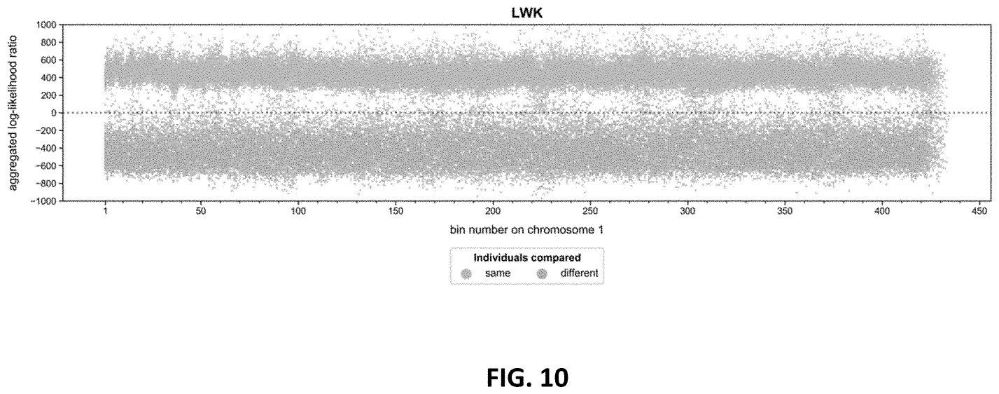

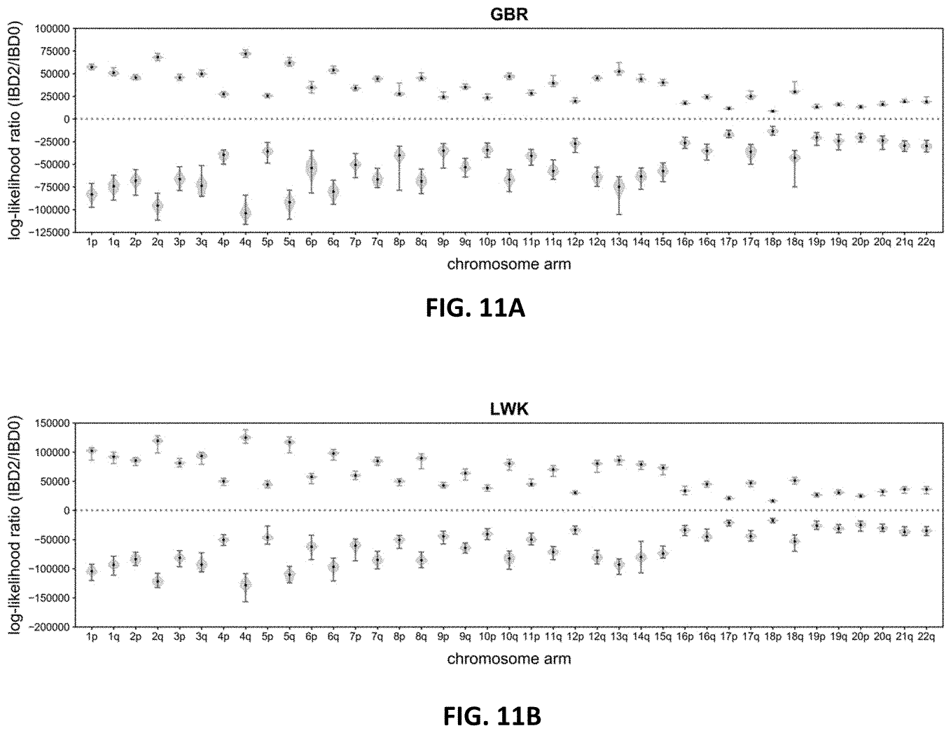

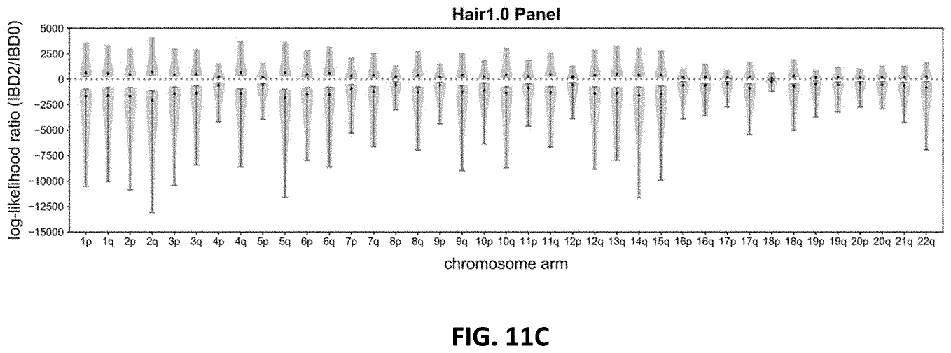

Additional sample comparison data is shown in FIGS. 8-11.

### Example 2—IBDGem with Genotype Array Data

The 1000 Genomes project pipeline calls variants from shotgun sequencing data across the genome. Each individual has genotype calls at nearly all sites that are found to be variable in the panel. Therefore, for each IBDGem analysis, the number of sites available for comparison is limited chiefly by the data available from the questioned sample. High-coverage genomic data can be used to generate nearly complete call sets at all of the sites known to be variable within humans in, for example, the 1000 Genomes panel. Thus, the genotype call set will include tens of millions of sites, although any specific individual will be homozygous for the reference allele at most of these sites.

In contrast, commercially available genotype arrays provide highly accurate genotype calls at about one million sites of known variation—those on the array—but no information at other sites. Genotype arrays are an accurate and less expensive approach for generating genotype data. To test the sensitivity of IBDGem when limited to data only at genotype array sites for the subject individual, the program was specified to perform comparisons on only bi-allelic sites found on the Illumina Global Screening Array (GSA). In both the GBR and LWK panels, it was found that for all self comparisons, the IBD2/IBD0 log-likelihood ratios remain higher than 100 and for all non-self comparisons, these ratios are less than −100 (FIG. 4). That is, IBDGem can compare data at only GSA array sites against 1-fold genome coverage DNA data and confidently discriminate self from non-self comparisons.

### Example 3—IBDGem Comparison with Data from Rootless Hairs

The sequencing libraries that generated the 1000 Genomes data were predominantly made from cell line derived, high-molecular weight DNA. In this way, the data quality is superior to what is possible from many forensic samples. To test the power of IBDGem using data derived from a more realistic forensic DNA source, DNA from rootless hairs from a panel of eight individuals was extracted and sequenced. Separately, DNA from the saliva of these same eight individuals was collected for genotype analysis using the Illumina Multi-Ethnic Global array.

Multiple head and pubic hairs from each individual were collected. DNA from each hair was extracted in 50 μL elution volume, and the two hair extracts with the highest DNA concentrations were chosen for sequencing. For many of the hair extracts, the DNA concentration was below the level of detection with qubit fluorimetry. Using 20 μL of each extract (40% of total volume), sequencing libraries were generated using a single-stranded library approach [16]. These libraries were pooled and roughly 60 million read pairs per library were generated.

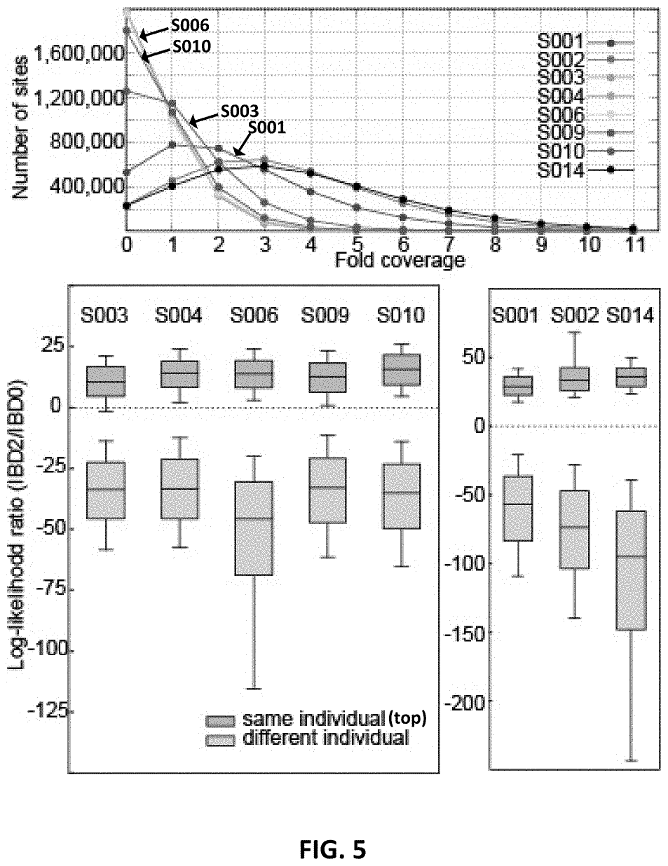

After mapping these sequence data to the reference human genome, it was found that the amount of usable human DNA for each hair sample was variable (FIG. 5—top). This is likely due to the variability of the amount of DNA present per unit length of hair amongst people [18].

Next, IBDGem was run, comparing each hair DNA dataset to each genotype dataset. For this comparison, the whole-panel 1000 Genomes allele frequencies were used as the background model since nothing about the donors was known and, as shown above, the method is largely insensitive to the use of a specific population background panel. All eight self comparisons and all 56 non-self comparisons were correctly identified (FIG. 5—bottom).

### Example 4—Relatedness Detection Using IBDGem

Determining self versus non-self using this framework is straightforward as self comparisons are IBD2 across every region of the genome and non-self comparisons are IBD0 across nearly every region. Closely related individuals, however, will share genomic regions where one chromosome is identical by descent (IBD1). Full siblings will also share some regions of IBD2.

To assess the power of IBDGem to detect regions of IBD1 and, more generally, to assess the degree of relatedness between compared samples, a module (sometimes referred to herein as “HiddenGem”) was implemented that finds the most likely path of the three IBD states through the genome using regional likelihood values of each state (FIG. 12). The family pedigrees present within the 1000 Genomes Phase 3 panel were used as the degrees of relatedness between individuals are known. While the IBD state (0, 1, or 2) is not known for any particular region of the genome, the total amount of each state is a simple function of the type of relatedness. For example, parent-child relatives must be IBD1 across the whole genome as the child inherits exactly one of their two chromosomes from each parent. On the other hand, full siblings are expected to share both parental chromosomes at one-quarter of the genome, neither parental chromosome at one-quarter of the genome, and one parental chromosome at one-half of the genome.

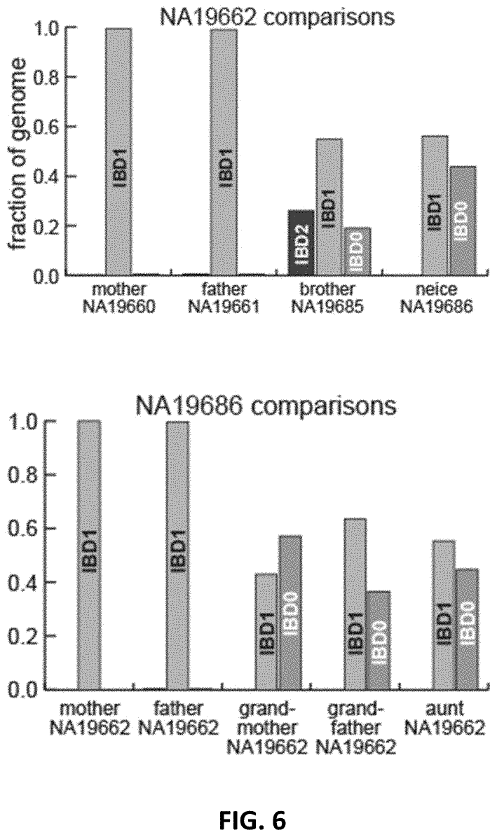

IBDGem was run followed by the maximum-likelihood IBD-state caller HiddenGem, comparing genotypes at only GSA sites for the known relatives of two individuals, NA19662 and NA19686, from the MXL (Mexican-American) population. In this experiment, the sequence data from each relative was down-sampled to one-fold average genome coverage. For each known relative, there is general concordance between the observed proportion of each IBD state and the expected values given the degree of relatedness (FIG. 6 and FIG. 8). Only the full-sibling relative comparison generates more than 1% of the genome assigned to IBD2. All parent-child comparisons assign all or nearly all of the genome to IBD1.

### Materials, Methods and Techniques

Data presented here are from: (1) The 1000 Genomes Project Phase 3 deep sequencing [15] and (2) a panel of eight human volunteers from whom DNA was derived from a saliva sample and cut hairs (hair panel).

For each anonymous study participant, saliva DNA, head hair, and pubic hair was collected. Saliva was collected using the OGR-500 collection device. 1 μg of extracted saliva DNA was submitted to AKESOgen for genotype array processing using the Illumina Multi-Ethnic Global Screening Array (MEGA). For each participant, DNA was extracted from 5 head and 3 pubic hairs, followed by preparation of single-stranded DNA Illumina sequencing libraries [16] from the two highest concentration head and pubic hair extractions. Libraries prepared from the hair extractions were sequenced on an Illumina NovaSeq 6000 at UCSF. Further details of the wet lab methods will now be provided.

Participants anonymously picked up and dropped off a collection kit containing an OGR-500 (DNA Genotek) saliva collection device, a plastic bag for head hair, a plastic bag for pubic hair, and a set of instructions. Each participant was requested to donate at least 5 head hairs, 3 pubic hairs, and saliva following the OGR-500 instructions.

For each saliva sample, DNA was extracted and submitted for array genotyping. First, DNA was extracted and isolated from 500 μL of each saliva sample using the prepIT-L2P (DNA Genotek) reagent, following the manufactures instructions. DNA was quantified using a Qubit dsDNA BR Assay Kit (Invitrogen) and a Qubit 4 fluorometer (Invitrogen). Next, 1 μg of DNA was submitted to Akesogen for genotyping on an Illumina MEGA array.

For each participant, 5 head hairs and 3 pubic hairs were trimmed and washed, followed by DNA extraction from the hairs. First, identifiable roots were removed and the head and pubic hairs were trimmed to a maximum of 5 cm and 3 cm, respectively. For external decontamination, each hair was submerged in 0.5% sodium hypochlorite for 10 seconds and then in three water baths for 10 seconds each. DNA was extracted and isolated from each hair using the Qiagen DNeasy Blood and Tissue Kit (Qiagen) following a user-developed protocol for hair [Purification of total DNA from nails, hair, or feathers using the DNeasy® Blood & Tissue Kit—(EN)]. DNA was eluted in 40 μL buffer EBT (10 mM Tris-HCl, 0.05% Tween). The DNA was quantified using a Qubit 1× dsDNA HS Assay Kit (Invitrogen) and a Qubit 4 fluorometer.

For each participant, Illumina sequencing libraries were prepared from the highest and lowest concentration head and pubic hair DNA extractions. First, 20 μL of each extract was concentrated to 11 μL using a SPRI bead mixture, which was prepared and performed as described in Rohland and Reich [Rohland, N. and D. Reich (2012) Genome Res. 22(5): p. 939-46] using 72 μL SPRI solution and a 108 μL isopropanol addition as described in Fishman et al [Fishman et al. (2018) Genome Biol. 19(1):113]. Next, Illumina libraries were prepared as described in Kapp et al. [J Hered. 2021. 112(3):241-249] with the following modifications: 10 μL of concentrated DNA extract, 1 μL 76 ng/μL ET SSB (NEB), 1 μL 2 μM P5 splinted adapter, 1 μL 0.4 μM P7 splinted adapter, and 12 μL reaction mix (38.33% PEG 8000, 104.16 mM Tris-HCl, 20.83 mM MgCl2, 20.83 mM DTT, 2.08 mM ATP, 41.66 U/μL T4 DNA Ligase (NEB), and 0.46 U/μL T4 PNK (NEB)). The libraries were purified using SPRI as described in Rohland and Reich [supra], beginning with the addition of 60 μL SPRI bead solution and 35 μL buffer EBT. The cleaned libraries were eluted in 20 μL buffer EBT.

Each library was amplified and double-indexed using the primers described in Kircher et al. [Nucleic Acids Res, 2012. 40(1):e3]. For each hair library, 50 μL reactions containing 20 μL library, 25 μL Amplitaq Gold 360 Master Mix (Applied Biosystems), 2.5 μL unique 20 μM i5 indexing primer, and 2.5 μL unique 20 μM i7 indexing primer were prepared. Each hair library was amplified with the following cycling conditions: 95° C. for 10 min, followed by 10 or 13 cycles of 95° C. for 30 s, 60° C. for 30 s, and 72° C. for 60 s, and a final extension of 72° C. for 7 min.

The post-amplified hair libraries were purified using SPRI ratios 1.2×. Next, each library was quantified using a Qubit 1×dsDNA HS Assay Kit and a Qubit 4 fluorometer, followed by visualization of each library using a D1000 ScreenTape (Agilent) and Tapestation 2200 (Agilent). Finally, all libraries were sequenced on one lane of NovaSeq using the S4 2×150 kit. The table below summarizes the data generated for each of the 4 libraries for each individual, the amount of human DNA that aligned to the reference genome (hg19) and the fold-coverage genome for each individual used in the IBDGem comparisons:

| | | | | | Genome |
|---|---|---|---|---|---|
| | | | | Unique | fold |
| Indi- | Hair | | | hg19 | cov- |
| vidual | type | Libraries | Read pairs | data (bp) | erage |
| S001 | Head | JK-H4-L2-S001 | 58903513 | 1,988,517,582 | 2.76 |
| | Head | JK-H5-L2-S001 | 73234840 | 2,327,240,175 | |
| | Pubic | JK-P1-L2-S001 | 62875789 | 2,424,276,772 | |
| | Pubic | JK-P2-L2-S001 | 51595063 | 1,553,184,598 | |
| S002 | Head | JK-H4-L2-S002 | 69597163 | 3,378,635,565 | 3.97 |
| | Head | JK-H5-L2-S002 | 61174383 | 2,709,910,139 | |
| | Pubic | JK-P1-L2-S002 | 61737394 | 3,444,859,236 | |
| | Pubic | JK-P3-L2-S002 | 55882601 | 2,375,310,070 | |
| S003 | Head | JK-H1-L2-S003 | 61370299 | 154,512,921 | 0.76 |
| | Head | JK-H5-L2-S003 | 63908772 | 197,672,430 | |
| | Pubic | JK-P1-L2-S003 | 70075886 | 892,140,476 | |
| | Pubic | JK-P2-L2-S003 | 68277093 | 1,026,517,519 | |
| S004 | Head | JK-H4-L2-S004 | 74359550 | 892,943,437 | 0.85 |
| | Head | JK-H5-L2-S004 | 47284158 | 217,373,308 | |
| | Pubic | JK-P1-L2-S004 | 59862426 | 939,239,134 | |
| | Pubic | JK-P2-L2-S004 | 47502026 | 485,806,890 | |
| S006 | Head | JK-H4-L2-S006 | 54388564 | 849,188,437 | 0.81 |
| | Head | JK-H5-L2-S006 | 60218154 | 686,290,100 | |
| | Pubic | JK-P1-L2-S006 | 54641178 | 403,574,821 | |
| | Pubic | JK-P2-L2-S006 | 57592900 | 487,350,961 | |
| S009 | Head | JK-H4-L2-S009 | 71357975 | 1,307,421,799 | 1.56 |
| | Head | JK-H5-L2-S009 | 61745891 | 891,957,093 | |
| | Pubic | JK-P1-L2-S009 | 60004101 | 1,195,248,050 | |
| | Pubic | JK-P2-L2-S009 | 69261852 | 1,276,147,255 | |
| S010 | Head | JK-H4-L2-S010 | 66312777 | 869,570,337 | 1.05 |
| | Head | JK-H5-L2-S010 | 51545404 | 742,650,430 | |
| | Pubic | JK-P1-L2-S010 | 58675747 | 644,505,735 | |
| | Pubic | JK-P2-L2-S010 | 59394449 | 890,243,781 | |
| S014 | Head | JK-H2-L2-S014 | 89974382 | 4,969,863,951 | 4.27 |
| | Head | JK-H4-L2-S014 | 81028214 | 5,008,444,003 | |
| | Pubic | JK-P1-L2-S014 | 42337258 | 814,204,627 | |
| | Pubic | JK-P2-L2-S014 | 49274060 | 2,012,826,232 | |

### Computational Details for Data Analysis

DNA sequence data from the hair panel were processed using SeqPrep [St. John, J. Seq Prep. Available from: github.com/jstjohn/SeqPrep]. Only read-pairs that were merged were used for downstream analysis. Merged reads were aligned to hs37d5.fa (hg19 human reference genome) using bwa aln with the following command set:

bwa aln -t 48 -l 26 hs37d5.fa LIB.fq>LIB.sai
bwa samse hs37d5.fa LIB.sai LIB.fa|
samtools view -Sb -o - |
samtools sort -o LIB.sorted.bam -

BAM files for each library were further processed with Picardtools CleanSam and MarkDuplicates. Then, BAM files from each individual were merged into a single bam file for downstream processing.

The merged BAM files were then filtered to remove any reads that are longer than 80 bp as DNA fragments from hair are rarely this long and the few DNA fragments longer than this cutoff show abnormally low concordance with genotype array data.

Since IBDGem takes in sequence data of single chromosomes in the pileup format, samtools mpileup was used to generate pileup files of all 22 autosomes from each individual's BAM file:

samtools mpileup -r CHROM -A -a -q 30 -Q 30 -s INDV.all-hair.180.bam -o INDV.all-hair.180.CHROM.pileup

IBDGem also takes in genotype data in the IMPUTE format. Therefore, a joint VCF from the genotype array data of all Hair1.0 individuals for each separate autosome was first created. Then, these files were further converted to the IMPUTE format using vcftools with the following options:

vcftools --vcf HAIR1-joint.CHROM.vcf \
--max-alleles 2
--min-alleles 2
--max-missing 1
--IMPUTE
--out HAIR1-joint.CHROM
--phased

This command generated a .legend, .indv, and .hap file for each chromosome, which were used as inputs to IBDGem.

Finally, the IBDGem comparison was performed between any two individuals on a specific chromosome with the following options:

ibdgem --hap HAIR1-joint.CHROM.hap \
--legend HAIR1-joint.CHROM.legend \
--indv HAIR1-joint.CHROM.indv \
--pileup INDV1.all-hair.180.CHROM.pileup \
--allele-freqs 1KG.GLOBAL.AF.dat \
--pileup-name INDV1 \
--sample INDV2 \
--variable-sites-only

For the GBR and LWK panels from 1000 Genomes, pileup files were generated for each unrelated individual using samtools mpileup with the same options as for the Hair1.0 panel. The joint VCF file for all 3,202 individuals was also converted to the IMPUTE format, removing known relatives with the following options:

vcftools --gzvcf 1KG-joint.CHROM.vcf.gz \
--IMPUTE \
--keep 1KG.unrelated.txt \
--out 1KG.unrelated.CHROM \
--remove-indels \
--max-missing 1 \
--max-alleles 2 \
--min-alleles 2 \
--mac 3 \
--remove-filtered-all

After relative filtering, a total of 2,594 individuals remained in the joint IMPUTE files.

IBDGem comparisons were then performed between any two individuals with the following options:

ibdgem --hap 1KG.unrelated.CHROM.hap \
--legend 1KG.unrelated.CHROM.legend \
--indv 1KG.unrelated.CHROM.indv \
--pileup INDV1.CHROM.pileup \
--pileup-name INDV1 \
--sample INDV2 \
--max-cov 60 \
--downsample-cov COV \
--variable-sites-only \

### REFERENCES

1. Kimpton, C. P., et al., Automated DNA profiling employing multiplex amplification of short tandem repeat loci. PCR Methods Appl, 1993. 3(1): p. 13-22.
2. Jobling, M. A. and P. Gill, Encoded evidence: DNA in forensic analysis. Nat Rev Genet, 2004. 5(10): p. 739-51.
3. Gill, P., A. J. Jeffreys, and D. J. Werrett, Forensic application of DNA ‘fingerprints’. Nature, 1985. 318(6046): p. 577-9.
4. Jeffreys, A. J., V. Wilson, and S. L. Thein, Individual-specific ‘fingerprints’ of human DNA. Nature, 1985. 316(6023): p. 76-9.
5. Genomes Project, C., et al., A global reference for human genetic variation. Nature, 2015. 526(7571): p. 68-74.
6. Browning, B. L. and S. R. Browning, Detecting identity by descent and estimating genotype error rates in sequence data. Am J Hum Genet, 2013. 93(5): p. 840-51.
7. Browning, B. L. and S. R. Browning, Improving the accuracy and efficiency of identity-by-descent detection in population data. Genetics, 2013. 194(2): p. 459-71.
8. Gusev, A., et al., Whole population, genome-wide mapping of hidden relatedness.

Genome Res, 2009. 19(2): p. 318-26.
9. Ball, C. A., et al., AncestryDNA Matching White Paper. AncestryDNA 2016.
10. Durand, E. Y., N. Eriksson, and C. Y. McLean, Reducing pervasive false-positive identical-by-descent segments detected by large-scale pedigree analysis. Mol Biol Evol, 2014. 31(8): p. 2212-22.
11. Alaeddini, R., S. J. Walsh, and A. Abbas, Forensic implications of genetic analyses from degraded DNA—a review. Forensic Sci Int Genet, 2010. 4(3): p. 148-57.
12. de Vries, J. H., et al., Impact of SNP microarray analysis of compromised DNA on kinship classification success in the context of investigative genetic genealogy. Forensic Sci Int Genet, 2022. 56: p. 102625.
13. Nielsen, R., et al., Genotype and SNP calling from next-generation sequencing data. Nat Rev Genet, 2011. 12(6): p. 443-51.
14. Swango, K. L., et al., A quantitative PCR assay for the assessment of DNA degradation in forensic samples. Forensic Sci Int, 2006. 158(1): p. 14-26.
15. Byrska-Bishop, M., et al., High coverage whole genome sequencing of the expanded 1000 Genomes Project cohort including 602 trios. bioRxiv, 2021.
16. Kapp, J. D., R. E. Green, and B. Shapiro, A Fast and Efficient Single-stranded Genomic Library Preparation Method Optimized for Ancient DNA. J Hered, 2021. 112(3): p. 241-249.
17. Rosenberg, N. A., et al., Genetic structure of human populations. Science, 2002. 298(5602): p. 2381-5.
18. Szabo, S., et al., In situ labeling of DNA reveals interindividual variation in nuclear DNA breakdown in hair and may be useful to predict success of forensic genotyping of hair. Int J Legal Med, 2012. 126(1): p. 63-70.
19. Vohr, S. H., et al., A phylogenetic approach for haplotype analysis of sequence data from complex mitochondrial mixtures. Forensic Sci Int Genet, 2017. 30: p. 93-105.
20. Vohr, S. H., et al., A method for positive forensic identification of samples from extremely low-coverage sequence data. BMC Genomics, 2015. 16: p. 1034.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A computer-implemented method for comparing genotype data from a first sample to a limited amount of DNA sequence data from a second sample, the method being implemented using one or more processors and one or more non-transitory computer-readable media comprising instructions stored thereon, which when executed by the one or more processors, cause the one or more processors to perform operations comprising:
(a) receiving genotype data from a first sample and a limited amount of DNA sequence data from a second sample;
(b) comparing the genotype data and limited amount of DNA sequence data at a plurality of variable sites across one or more genomic regions;
(c) at each of the plurality of variable sites, calculating the likelihood of the limited amount of DNA sequence data and the genotype data being related under at least two models of relatedness, wherein the at least two models of relatedness comprise:
(i) the first sample and the second sample share two chromosomes identical-by-descent (IBD2); and
(ii) the first sample and the second sample share no chromosomes identical-by-descent (IBD0);
(d) at each of the plurality of variable sites, comparing the likelihood of model (i) to the likelihood of model (ii).

2. The computer-implemented method according to claim 1, wherein comparing the likelihood of model (i) to the likelihood of model (ii) comprises generating a log-likelihood ratio of model (i) and model (ii), thereby generating a plurality of log-likelihood ratios comprising a log-likelihood ratio for each of the plurality of variable sites.

3. The computer-implemented method according to claim 2, further comprising aggregating the plurality of log-likelihood ratios.

4. The computer-implemented method according to claim 3, wherein log-likelihood ratios are aggregated across each arm of each autosome.

5. The computer-implemented method according to claim 1, wherein the first sample is from a known individual and the second sample is an unknown sample.

6. The computer-implemented method according to claim 5, further comprising determining whether the unknown sample is from the known individual.

7. The computer-implemented method according to claim 6, further comprising aggregating the plurality of log-likelihood ratios, and wherein determining whether the unknown sample is from the known individual is based on the aggregated log-likelihood ratios, optionally wherein the plurality of log-likelihood ratios are aggregated across each arm of each autosome.

8. The computer-implemented method according to claim 1, wherein receiving the genotype data comprises receiving a VCF file comprising the genotype data.

9. The computer-implemented method according to claim 1, wherein the genotype data was generated by massively parallel sequencing (MPS).

10. The computer-implemented method according to claim 1, wherein the genotype data was generated by genotype array.

11. The computer-implemented method according to claim 1, wherein receiving the sequence data comprises receiving a BAM file comprising the sequence data.

12. The computer-implemented method according to claim 1, wherein the variable sites comprise single-nucleotide polymorphisms (SNPs), insertion-deletions (INDELs), or a combination thereof.

13. The computer-implemented method according to claim 1, wherein the second sample is a hair sample, a bone sample, a blood sample, a semen sample, or any combination thereof.

14. The computer-implemented method according to claim 13, wherein the hair sample is a rootless hair sample.

15. The computer-implemented method according to claim 14, wherein the hair sample is a single rootless hair sample.

16. The computer-implemented method according to claim 1, wherein the second sample was collected from a crime scene.

17. The computer-implemented method according to claim 1, wherein the first sample is from a person of interest in a criminal investigation.

18. The computer-implemented method according to claim 16, wherein the method is performed for a forensic analysis.

19. The computer-implemented method according to claim 1, wherein the limited amount of DNA sequence data comprises less than 2-fold genome coverage, less than 1.5-fold genome coverage, less than 1-fold genome coverage, less than 0.5-fold genome coverage, less than 0.1-fold genome coverage, or less than 0.05-fold genome coverage.

20. The computer-implemented method according to claim 1, wherein the limited amount of DNA sequence data was obtained from a sample comprising less than 1 nanogram of genomic DNA.

21. One or more non-transitory computer-readable media comprising instructions stored thereon, which when executed by one or more processors, cause the one or more processors to perform operations comprising:
(a) receiving genotype data from a first sample and a limited amount of DNA sequence data from a second sample;
(b) comparing the genotype data and limited amount of DNA sequence data at a plurality of variable sites across one or more genomic regions;
(c) at each of the plurality of variable sites, calculating the likelihood of the limited amount of DNA sequence data and the genotype data being related under at least two models of relatedness, wherein the at least two models of relatedness comprise:
(i) the first sample and the second sample share two chromosomes identical-by-descent (IBD2); and
(ii) the first sample and the second sample share no chromosomes identical-by-descent (IBD0);
(d) at each of the plurality of variable sites, comparing the likelihood of model (i) to the likelihood of model (ii).

22. The one or more non-transitory computer-readable media of claim 21, wherein comparing the likelihood of model (i) to the likelihood of model (ii) comprises generating a log-likelihood ratio of model (i) and model (ii), thereby generating a plurality of log-likelihood ratios comprising a log-likelihood ratio for each of the plurality of variable sites.

23. The one or more non-transitory computer-readable media of claim 22, wherein the operations further comprise aggregating the plurality of log-likelihood ratios.

24. The one or more non-transitory computer-readable media of claim 23, wherein log-likelihood ratios are aggregated across each arm of each autosome.

25. The one or more non-transitory computer-readable media of claim 21, wherein the first sample is from a known individual and the second sample is an unknown sample.

26. The one or more non-transitory computer-readable media of claim 25, wherein the operations further comprise determining whether the unknown sample is from the known individual based on the aggregated log-likelihood ratios.

27. One or more non-transitory computer-readable media comprising instructions stored thereon, which when executed by one or more processors, cause the one or more processors to perform the computer-implemented method according to claim 1.

28. A computer system comprising the one or more non-transitory computer-readable media of claim 21.

29. A computer-implemented method for assessing the degree of relatedness between genotype data from a first sample and a limited amount of DNA sequence data from a second sample, the method being implemented using one or more processors and one or more non-transitory computer-readable media comprising instructions stored thereon, which when executed by the one or more processors, cause the one or more processors to perform operations comprising:
(a) receiving genotype data from a first sample from a first individual and a limited amount of DNA sequence data from a second sample from a second individual;
(b) comparing the genotype data and limited amount of DNA sequence data at a plurality of variable sites across one or more genomic regions;
(c) at each of the plurality of variable sites, calculating the likelihood of the limited amount of DNA sequence data and the genotype data being related under the following models of relatedness:
(i) the first sample and the second sample share two chromosomes identical-by-descent (IBD2);
(ii) the first sample and the second sample share one chromosome identical-by-descent (IBD1); and
(iii) the first sample and the second sample share no chromosomes identical-by-descent (IBD0); and
(d) determining the most likely path of the three IBD models through the one or more genomic regions using regional likelihood values of each model.

30. The computer-implemented method according to claim 29, wherein the genotype data from the first sample is available from an online database.

31. The computer-implemented method according to claim 29, wherein step (d) is performed using a dynamic programming algorithm.

32. The computer-implemented method according to claim 31, wherein the dynamic programming algorithm comprises a forward algorithm, a forward-backward algorithm, or a Viterbi algorithm.

33. The computer-implemented method according to claim 29, wherein the operations further comprise partitioning the most likely path into IBD2, IBD1 and IBD0.

34. The computer-implemented method according to claim 29, wherein the operations further comprise identifying regions of co-inheritance for IBD2, IBD1 and/or IBD0.

35. The computer-implemented method according to claim 29, wherein two individuals are related to the second individual, and wherein the operations further comprise identifying IBD1 segments that are shared between the two individuals but not shared between the two individuals and the second individual, thereby identifying IBD1 segments from a common ancestor of the two individuals other than the second individual.

36. The computer-implemented method according to claim 29, further comprising, based on the determination at step (d), identifying the first and second individuals as parent-child relatives.

37. The computer-implemented method according to claim 29, further comprising, based on the determination at step (d), identifying the first and second individuals as full siblings.

38. One or more non-transitory computer-readable media comprising instructions stored thereon, which when executed by one or more processors, cause the one or more processors to perform operations comprising:
(a) receiving genotype data from a first sample from a first individual and a limited amount of DNA sequence data from a second sample from a second individual;
(b) comparing the genotype data and limited amount of DNA sequence data at a plurality of variable sites across one or more genomic regions;
(c) at each of the plurality of variable sites, calculating the likelihood of the limited amount of DNA sequence data and the genotype data being related under the following models of relatedness:
(i) the first sample and the second sample share two chromosomes identical-by-descent (IBD2);
(ii) the first sample and the second sample share one chromosome identical-by-descent (IBD1); and
(iii) the first sample and the second sample share no chromosomes identical-by-descent (IBD0); and
(d) determining the most likely path of the three IBD states through the one or more genomic regions using regional likelihood values of each model.

39. One or more non-transitory computer-readable media comprising instructions stored thereon, which when executed by one or more processors, cause the one or more processors to perform the computer-implemented method according to claim 29.

40. A computer system comprising the one or more non-transitory computer-readable media of claim 39.
